(19) United States
(12) Patent Application Publication
(10) Pub. No.: US 2018/0280332 A1
(43) Pub. Date: Oct. 4, 2018

(54) METHOD FOR IMPROVING EQUILIBRIOCEPTION IN HEALTHY INDIVIDUALS AND NUTRITIONAL COMPOSITION

(71) Applicant: N.V. Nutricia, Zoetermeer (NL)

(72) Inventors: Mattheus Cornelis DE WILDE, Utrecht (NL); Danielle Stefanie COUNOTTE, Utrecht (NL)

(73) Assignee: N.V. Nutricia, Zoetermeer, (NL)

(21) Appl. No.: 15/770,109

(22) PCT Filed: Oct. 24, 2016
(86) PCT No.: PCT/NL2016/050734

§ 371 Date: Apr. 20, 2018

(30) Foreign Application Priority Data
Oct. 23, 2015 (NL) PCT/NL2015/050736

(51) Int. Cl. A61K 31/202; A23L 33/12; A23L 33/175; A23L 33/155; A23L 33/13; A61P 27/00; A61K 31/675; A61K 31/714; A61K 31/14; A61K 31/198; A61K 31/4172; A61K 33/04; A61K 31/375; A61K 31/355

(52) U.S. Cl. CPC A61K 31/202; A23V 2002/00; A23L 33/175; A23L 33/155; A23L 33/13; A61P 27/00; A61K 31/675; A61K 31/714; A61K 31/14; A61K 31/198; A61K 31/4172; A61K 33/04; A61K 31/375; A61K 31/355; A23L 33/12

(57) ABSTRACT

The invention pertains to a method for improving or promoting equilibrioception or coordination and balance in a healthy subject, comprising administration of: (i) ω-3 polyunsaturated fatty acid (LCPUFA) selected from the group consisting of docosahexaenoic acid (DHA), eicosapentaenoic acid (EPA) and docosapentaenoic acid (DPA); (ii) one of more of uridine, cytidine and/or an equivalent thereof, including salts, phosphates, acyl derivatives and/or esters; and (iii) free leucine, to said healthy subject. The invention also pertains to a composition for use in such a method.

The invention is in the field of nutrition and more particularly relates to nutritional compositions for use in improving equilibrioception in healthy individuals. The invention relates to the use of specific amino acids, omega-3 polyunsaturated fatty acids and a uridine source in the improvement of a subject's coordination and balance.

### BACKGROUND TO THE INVENTION

Equilibrioception is a physiological sense that helps subjects to maintain balance and prevents them from falling over. Healthy people can benefit from improvement in coordination and balance in their daily life activities and duties. WO 2015/084161 discloses a combination of a uridine source and omega-3 polyunsaturated fatty acids for prevention or treatment of specific disturbances in coordination of limbs and disturbances in equilibrium in a mammal. It relates to diseases, disorders and problems with proper functioning of the brain, such as neurodegenerative diseases.

### SUMMARY OF THE INVENTION

The inventors found that equilibrioception of healthy subjects surprisingly improved upon administration of a combination of (i) uridine, cytidine and/or equivalents thereof, (ii) long chain omega-3 polyunsaturated fatty acids preferably comprising at least DHA; and (iii) free leucine. As demonstrated in the examples, the combination according to the invention significantly improved the performance of healthy subjects in a balance beam experiment. Without being bound to a theory, it is believed that the combination helps reducing phenylalanine levels in the brain, or preventing increases of brain phenylalanine levels, and consequently improve a subject's equilibrioception or a subject's coordination and balance.

In view of these findings, the invention pertains to the use of a composition in the manufacture of a product for improving or promoting equilibrioception or coordination, and balance in a healthy subject. The invention also pertains to a composition, combination of product for (non-therapeutic) use in improving or promoting equilibrioception or coordination and balance in a healthy subject. The invention also relates to a (non-therapeutic) method for improving or promoting equilibrioception or coordination and balance in a healthy subject, said method comprising administering a composition, combination or product to a healthy subject.

The composition, combination or productcomprises (i) ω-3 polyunsaturated fatty acid (LCPUFA) selected from the group consisting of docosahexaenoic acid (DHA), eicosapentaenoic acid (EPA) and docosapentaenoic acid (DPA); (ii) one of more of uridine, cytidine and/or an equivalent thereof, including salts, phosphates, acyl derivatives and/or esters; and (iii) free leucine.

### LIST OF PREFERRED EMBODIMENTS

1. A method for improving or promoting equilibrioception or coordination and balance in a healthy subject, comprising administration of: (i) ω-3 polyunsaturated fatty acid (LCPUFA) selected from the group consisting of docosahexaenoic acid (DHA), eicosapentaenoic acid (EPA) and docosapentaenoic acid (DPA); (ii) one of more of uridine, cytidine and/or an equivalent thereof, including salts, phosphates, acyl derivatives and/or esters; and (iii) free leucine, to said healthy subject.
2. The method according to embodiment 1, wherein the subject is a healthy human subject.
3. The method according to any of the preceding embodiments, wherein (iii) comprises threonine, valine, histidine and methionine, wherein the sum of threonine, valine, histidine and methionine is at least 12 wt %, preferably at least 13 wt %, more preferably at least 14 wt %, preferably 15-60 wt %, most preferably 16-40 wt % based on total proteinaceous matter.
4. The method according to any of the preceding embodiments, wherein (iii) comprises leucine, wherein the total amount of L-leucine provided by the proteinaceous matter amounts to at least 7 wt %, preferably at least 8 wt %, more preferably 9-20 wt %, most preferably 9-15 wt %, based on total proteinaceous matter.
5. The method according to any of the preceding embodiments, wherein (iii) is essentially free of phenylalanine (Phe).
6. The method according to any of the preceding embodiments, wherein (iii) comprises leucine, isoleucine and valine, and said amino acids altogether are present in an amount of at least 15 wt %, more preferably between 15 and 35 wt %, even more preferably between 16 and 30 wt % based on total proteinaceous matter.
7. The method according to any of the preceding embodiments, further comprising administering vitamin D and/or vitamin K, or functional equivalents thereof.
8. The method according to any of the preceding embodiments, further comprising administering at least one of the B vitamins selected from the group consisting of vitamin B6, vitamin B12 and vitamin B9, including functional equivalents thereof, preferably B6 and/or B12.
9. The method according to any of the preceding embodiments, further comprising administering choline, a choline salt and/or choline ester.
10. The method according to any of the preceding embodiments, further comprising administering at least one, preferably at least two, most preferably all of the antioxidants selected from the group consisting of selenium, vitamin C and vitamin E, and functional equivalents thereof, preferably at least selenium.
11. A nutritional composition comprising (i) ω-3 polyunsaturated fatty acid (LCPUFA) selected from the group consisting of docosahexaenoic acid (DHA), eicosapentaenoic acid (EPA) and docosapentaenoic acid (DPA); (ii) one of more of uridine, cytidine and/or an equivalent thereof, including salts, phosphates, acyl derivatives and/or esters; and (iii) free leucine, wherein the total amount of leucine provided by the proteinaceous matter amounts to at least 7 wt %, preferably at least 8 wt %, more preferably 9-20 wt %, most preferably 9-15 wt %, based on total proteinaceous matter, and wherein the composition is essentially free from Phe.
12. The composition according to embodiment 11, further comprising threonine, valine, histidine and methionine, wherein the sum of threonine, valine, histidine and methionine is at least 12 wt %, preferably at least 13 wt %, more preferably at least 14 wt %, preferably 15-60 wt %, most preferably 16-40 wt % based on total proteinaceous matter.
13. The composition according to embodiment 11 or 12, comprising leucine, isoleucine and valine, and said amino acids altogether are present in an amount of at least 15 wt %, more preferably between 15 and 35 wt %, even more preferably between 16 and 30 wt % based on total proteinaceous matter.
14. The composition according to any one of embodiments 11-13, further comprising vitamin D and/or vitamin K, or functional equivalents thereof.
15. The composition according to any one of embodiments 11-14, further comprising at least one of the B vitamins selected from the group consisting of vitamin B6, vitamin B12 and vitamin B9, including functional equivalents thereof, preferably vitamin B6 and/or B12.
16. The composition according to any one of embodiments 11-15, further comprising choline, a choline salt and/or choline ester.
17. The composition according to any one of embodiments 11-16, further comprising at least one, preferably at least two, most preferably all of the antioxidants selected from the group consisting of selenium, vitamin C and vitamin E, and functional equivalents thereof, preferably at least selenium.
18. Use of (i) ω-3 polyunsaturated fatty acid (LCPUFA) selected from the group consisting of docosahexaenoic acid (DHA), eicosapentaenoic acid (EPA) and docosapentaenoic acid (DPA); (ii) one of more of uridine, cytidine and/or an equivalent thereof, including salts, phosphates, acyl derivatives and/or esters; and (iii) free leucine, for improving equilibrioception, coordination and/or balance.

### LIST OF FIGURES

FIGS. 1A and 1B show a schematic representation of a correct and incorrect step, respectively, of a mouse moving on a rod;

FIG. 2 shows the intervention effect on healthy mice in a balance beam experiment.

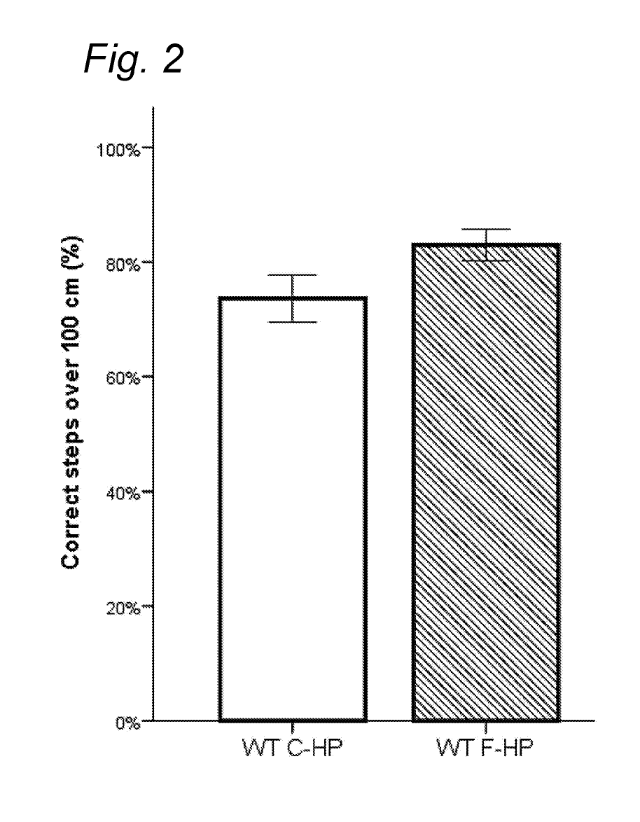

### DETAILED DESCRIPTION OF THE INVENTION

The invention pertains to a (non-therapeutic) method for improving or promoting equilibrioception or coordination and balance in a healthy subject, said method comprising administering a composition, combination or product to a healthy subject, comprising said composition comprising (i) ω-3 polyunsaturated fatty acid (LCPUFA) selected from the group consisting of docosahexaenoic acid (DHA), eicosapentaenoic acid (EPA) and docosapentaenoic acid (DPA); (ii) one of more of uridine, cytidine and/or an equivalent thereof, including salts, phosphates, acyl derivatives and/or esters; and (iii) free leucine.

In the context of the present invention, “equilibrioception” could also be worded as “sense of balance”. The present use or method is thus for improving equilibrioception, which may also be worded as “promoting equilibrioception”, “supporting equilibrioception” or “stimulating equilibrioception”. Equilibrioception can be assessed through a balancing ability test in a mammal (under lab conditions), and thus also to test whether a certain treatment has an effect on improving or enhancing equilibrium, using a rotarod test as described in the experimental section.

Advantageously, the method or use according to the invention is for improving equilibrioception in healthy subjects. In one embodiments, the invention pertains to subjects not suffering from a balance dysfunction, or known to be at risk of developing such balance dysfunction. The inventors surprisingly found that in healthy subjects equilibrioception could be improved upon administration of the combination of the invention. In one aspect, the method or use according to the invention is thus characterized as non-medical.

The equilibrioception of a subject is improved upon administration of the combination according to the invention. In the context of the present invention, the subject is preferably a human subject. In the context of the present invention, the subject is preferably a healthy subject. Most preferably, the subject is a healthy human subject, preferably non-elderly (preferably less than 50 years of age).

The composition, method and combination will be outlined in more detail here below. In the context of defining the actives according to the present invention, the terms “product”, “composition” and “combination” are used interchangeably.

### ω-3 LC-PUFAs

The composition or combination of the invention preferably comprises at least one ω-3 long-chain polyunsaturated fatty acid (LC PUFA; having a chain length of 18 and more carbon atoms) selected from the group consisting of docosahexaenoic acid (22:6; DHA), eicosapentaenoic acid (20:5; EPA) and docosapentaenoic acid (22:5 ω-3; DPA), preferably at least one of DHA and EPA. Preferably the present composition or combination contains at least DHA, more preferably DHA and EPA. EPA is converted to DPA (ω-3), increasing subsequent conversion of DPA to DHA in the brain. Hence, the present composition or combination preferably also contains a significant amount of EPA, so to further stimulate in vivo DHA formation.

The LCPUFAs (DHA, EPA and/or DPA) are preferably provided as triglycerides, diglycerides, monoglycerides, free fatty acids or their salts or esters, phospholipids, lysophospholipids, glycerol ethers, lipoproteins, ceramides, glycolipids or combinations thereof. Preferably, the present composition or combination comprises at least DHA in triglyceride form. Suitable ω-3 LCPUFA and/or DHA sources include tuna oil, (other) fish oils, DHA-rich alkyl esters, algae oil, egg yolk, or phospholipids enriched with ω-3 LCPUFA e.g. phosphatidylserine-DHA. Preferably, a composition or combination according to the invention comprises fish oil providing the omega-3 LCPUFA(s), Another particularly suitable source for the omega-3 LCPUFA(s) is algae oil.

If EPA, DHA and/or EPA are present, the total daily dosage of DHA+EPA+DPA taken together is in the range of 0.25 to 5 g per day, more preferably 0.5 to 5 g per day, most preferably 1 to 2.5 g per day. In a preferred embodiment, these amounts are based on the total sum of DHA and EPA if present. DHA is preferably administered in an amount of at least 0.5 g per day, more preferably 0.5 to 2.5 g per day, most preferably at least 1 g per day.

In terms of the composition, combination or method, the proportion of ω-3 LCPUFA (more preferably DHA+EPA+DPA, most preferably DHA+EPA) of the total fatty acids in the composition is preferably 5 to 95 wt %, more preferably 10 to 80 wt %, most preferably 15 to 70 wt %, even more preferably 20 to 60 wt % of the total fatty acids. The present composition or combination preferably comprises 5 to 95 wt % DHA based on total fatty acids, preferably 10 to 75 wt % DHA based on total fatty acids, more preferably 10 to 60 wt %, even more preferably 10-50 wt %, more preferably 10-40 wt %, especially at least 20 wt % DHA, based on total fatty acids of the composition or combination. The present composition or combination preferably comprises 5 to 95 wt % EPA based on total fatty acids, preferably 5 to 75 wt % EPA, even more preferably 5-50 wt %, more preferably 5-25 wt %, most preferably 5-15 wt %, based on total fatty acids of the composition or combination. In terms of DHA content in a composition or combination in accordance with the present invention, the DHA content is preferably 0.5-1.5 g per 100 ml of the (liquid) product. The amount of EPA is preferably at least 0.1 g, more preferably at least 0.2 g of the product. The above-mentioned amounts take into account and optimize several aspects, including taste (e.g. too high LCP levels reduce taste, resulting in a reduced compliance).

In the method, combination or composition of the invention, the ratio of the weight of DHA to EPA is preferably larger than 1, more preferably 2:1 to 10:1, more preferably 2:1 to 5:1. The ratios take into account and optimize the balance between DHA and precursors thereof to ensure optimal effectiveness while maintaining low-volume formulations.

If arachidonic acid (AA) is present or administered, it concerns a very low amount of AA, expressed in terms of a DHA/AA weight ratio in the present composition, combination or method of at least 5, preferably at least 10, more preferably at least 15, preferably at least 20, most preferably at least 25. If AA is administered, it preferably amounts to less than 5 weight %, more preferably less than 2.5 weight %, preferably less than 1 wt % based on total fatty acids of the composition or combination.

Uridine, Cytidine and/or Equivalents Thereof

The method, combination and composition according to the invention preferably comprise one or more of uridine, cytidine and/or an equivalent thereof, including salts, phosphates, acyl derivatives and/or esters. The method, combination and composition preferably comprises at least one uridine or an equivalent thereof selected from the group consisting of uridine (i.e. ribosyl uracil), deoxyuridine (deoxyribosyl uracil), uridine phosphates (UMP, dUMP, UDP, UTP), nucleobase uracil and acylated uridine derivatives. In one embodiment, cytidine, CMP, citicoline (CDP-choline) may also be applied in addition to or instead of uridine (equivalent). Preferably, the composition or combination to be administered according to the present invention comprises a source of uridine selected from the group consisting of uridine, deoxyuridine, uridine phosphates, uracil, and acylated uridine.

Preferably, the method, combination and composition according to the invention comprise an uridine phosphate selected from the group consisting of uridine monophosphate (UMP), uridine diphosphate (UDP) and uridine triphosphate (UTP); and/or a cytidine phosphate (CMP, CDP, CTP, preferably CMP). In a preferred embodiment, the composition or combination comprises at least one of the aforementioned uridine phosphates. Most preferably the present composition or combination comprises UMP, as UMP is most efficiently being taken up by the body. Hence, inclusion of UMP in the present method, combination and composition enables a high effectivity or efficacy at the lowest dosage and/or the administration of a low volume to the subject. Preferably at least 50 weight % of the uridine in the present method, combination and composition is provided by UMP, more preferably at least 75 weight %, most preferably at least 95 weight %. Doses administered are given as UMP. The amount of uracil sources can be calculated taking the molar equivalent to the UMP amount (molecular weight 324 Dalton).

The present method preferably comprises the administration of uridine (the cumulative amount of uridine, deoxyuridine, uridine phosphates, nucleobase uracil and acylated uridine derivatives) in an amount of 0.05-5 g per day, preferably 0.1-2.5 g per day, more preferably 0.25-1 g per day. The present method preferably comprises the administration of a composition or combination comprising uridine in an amount of 0.05-5 g UMP per 100 ml liquid product, preferably 0.1-2.5 g UMP per 100 ml liquid product, more preferably 0.25-1 g per 100 ml liquid product. In one embodiment, the present method, combination and composition may comprise the administration a uridine source in a concentration of 0.4 mg-3000 mg, calculated as UMP, per 100 kcal, preferably 0.6 mg-2000 mg, calculated as UMP, per 100 kcal product, more preferably 1 mg-1000 mg, calculated as UMP per 100 kcal product. The terms product, composition and combination are used interchangeably. Preferably 1-40 mg UMP per kilogram body weight is administered per day, more preferably 5-35, even more preferably 5-30 mg UMP/kg body weight. The above amounts also account for any amounts of cytidine, cytidine phosphates and citicoline incorporated in the composition, combination or method.

Amino acids

The combination of the invention contains free amino acids. According to the present invention, “free amino acids” are amino acids not coupled to other amino acids, but the term includes amino acids salts or di- and tripeptides such as cystine or gly-gly dipeptides. Next to free amino acids, other proteinaceous material may also be present in the combination according to the invention.

The composition, method and combination of the invention is preferably ‘substantially devoid from Phe’ or ‘essentially free of phenylalanine’, meaning that less than 5 wt %, preferably less than 2 wt % phenylalanine, more preferably less than 1 wt % Phe, more preferably less than 0.5 wt % Phe, even more preferably less than 0.1 wt % Phe, most preferably less than 0.05 wt % Phe, based on the total proteinaceous content of the composition or combination, is present. In order to achieve such low Phe levels, a product according to the invention comprises proteinaceous matter comprising free amino acids (other than phenylalanine) and/or a non-allergenic protein source such as glycomacropeptide (GMP). According to the present invention, ‘free amino acids’ are amino acids not coupled to other amino acids, but the term includes amino acids salts or di- and tripeptides such as cystine or gly-gly dipeptides. GMP is a protein originating from casein and is low in phenylalanine, and can improve the taste significantly without significantly increasing the phenylalanine content of the composition or combination. If the composition or combination comprises a protein fraction, it is preferred to involve a mixture of GMP supplemented with free amino acids to the extent desired and/or according to nutritional requirements. The composition or combination preferably comprises at least 50 wt %, preferably 70-90 wt % GMP based on the total protein content, preferably supplemented to 100% with free amino acids.

The combination according to the invention comprises a protein fraction, which is preferably predominant in free amino acids, but preferably low in Phe. It is preferred that the complete protein fraction of the combination according to the invention comprises 7-100 wt %, preferably 8-100 wt %, more preferably 9-100 wt %, more preferably 10-100 wt % preferably 50-95 wt % free amino acids.

A preferred amino acid composition according to the present invention has a relatively high content of the branched chain amino acids (BCAA) leucine, isoleucine and valine. These amino acids can potentially block the transport of phenylalanine over the intestinal barrier and also over the blood-brain barrier thereby helping in lowering the levels of phenylalanine in the brain, but without wishing to be tied down to any theory, the inventors also believe that adding these amino acids would increase cerebral protein synthesis. The protein fraction preferably comprises at least 15 wt % of said branched chain amino acids (BCAA), more preferably between 15 and 35 wt %, even more preferably between 16 and 30 wt %, particularly 17-25 wt %, most preferably at least 18 wt % based on the total protein content. These amino acids are preferably provided in free form.

The method, composition or combination of the invention comprises L-leucine in free form, wherein the total amount of L-leucine provided by the proteinaceous matter preferably amounts to at least 7 wt %, preferably at least 8 wt %, more preferably 9-20 wt %, most preferably 9-15 wt %, based on total proteinaceous matter. In the context of the invention, the terms leucine' and 1-leucine' are used interchangeably.

In order to improve LAT1-mediated blood-brain barrier transport of large neutral amino acids (LNAAs), it is preferred that the sum of threonine, valine, histidine and methionine at least 12 wt %, preferably at least 13 wt %, more preferably at least 14 wt %, even more preferably at least 15-60 wt %, more preferably 16-40 wt %, based on total proteinaceous matter (i.e. the sum of all amino acids, peptides and proteins and hydrolysates thereof). These amino acids are preferably provided in free form.

In particular, a nutritional composition according to the invention may comprise one or more amino acids, preferably at least the essential amino acids other than phenylalanine, more preferably all amino acids in a relative amount given in Table 1, per 100 g dry weight of the composition.

TABLE 1

| | Preferred range |
|---|---|
| Amino Acids | g/100 g dry weight |
| Alanine | 0.5-4.0 |
| Arginine | 1.0-7.0 |
| Aspartic acid | 0.8-6.5 |
| Cysteine | 0.4-3.5 |
| Glutamine | 1.0-5.0 |
| Glycine | 0.8-6.0 |
| Histidine | 0.5-4 |
| Iso-leucine | 0.5-6.0 |
| Leucine | 1.5-10 |
| Lysine | 1-7 |
| Methionine | 0.2-2 |
| Phenylalanine | 0-0.3 |
| Proline | 1-8 |
| Serine | 0.5-5 |
| Threonine | 0.6-6 |
| Tryptophan | 0.2-2.5 |
| Tyrosine | 1.0-9.0 |
| Valine | 1.0-7.0 |

The combination of the invention may comprise further components, such as vitamin D, vitamin K, choline, phospholipids, B vitamins, antioxidants. Preferably, the combination comprises at least 2, more preferably at least 3 of the aforementioned further components. Further details are given below.

Vitamin D, vitamin K

The method, composition or combination of the invention preferably comprises at least one of vitamins K and vitamin D and/or their functional equivalents, preferably both in therapeutically effective amounts. Within the context of the present invention, ‘vitamin D’ is understood to encompass vitamin D2 (ergocalciferol), vitamin D3 (cholecalciferol). Vitamin D, or a functional equivalent thereof, is preferably present in the method, composition or combination of the invention in an amount to provide a daily dosage in the range of 1 to 100 μg, in particular in the range of 1 to 50 μg, preferably 2-25 μg, preferably at least 3 μg, more preferably at least 4 μg, even more preferably at least 5 μg, more in particular in the range of 5 to 25 μg, even more preferably at least 5-15 μg. In one embodiment, vitamin D, or a functional equivalent thereof, is present in an amount in the range of 1 to 100 μg, in particular in the range of 2 to 50 μg, preferably 2-25 μg, preferably at least 3 μg, more preferably at least 4 μg, even more preferably at least 5 μg, more in particular in the range of 5 to 25 μg, even more preferably at least 5-15 μg per 100 ml of the (liquid) product. In the context of the invention, 1 IU of vitamin D is the biological equivalent of 0.025 μg. Hence, 1,000 IU is the biological equivalent of 25 μg.

The method, combination or composition according to the invention comprises at least a therapeutically effective amount of vitamin K, including vitamin K1 and vitamin K2. Vitamin K₂, also known as “menatetrenone”, “menaquinone-7”, “menaquinone” or “menadione”, is a group name for a family of related compounds, generally subdivided into short-chain menaquinones, with menatetrenone (“MK-4”) as the most important member, and long-chain menaquinones, of which MK-7, MK-8 and MK-9 are nutritionally the most recognized. Within the context of the present invention, ‘vitamin K, or a functional equivalent thereof’ is understood to refer to vitamin K1, vitamin K2, menaquinone-4 (MK-4), menaquinone-7 (MK-7). It is preferred that in the composition, Vitamin K, or a functional equivalent thereof, is present in an amount to provide a daily dosage in the range of 1 to 100 in particular in the range of 5 to 50 more in particular at least 7 μg, preferably at least 8 μg, more preferably at least 9 μg, even more preferably in the range of 10 to 50 μg, particularly at least 11, 12, 13, 14, 15 μg, preferably up to 40 μg, more preferably up to 30 μg. In one embodiment, vitamin K, or a functional equivalent thereof, is present in an amount in the range 1 to 100 μg, in particular in the range of 5 to 50 μg, at least 7 μg, preferably at least 8 μg, more preferably at least 9 μg, even more preferably in the range of 10 to 50 μg, particularly at least 11, 12, 13, 14, 15 μg, preferably up to 40 μg, more preferably up to 30 μg per 100 ml of the (liquid) product. Preferably vitamin K is present as vitamin K1.

### Choline

In a preferred embodiment, the method, combination and composition according to the present invention comprise choline, a choline salt and/or choline ester. Herein, the term ‘choline’ shall be considered to encompass all these equivalents. The choline salt is preferably selected from choline chloride, choline bitartrate, or choline stearate. The choline ester is preferably selected from the group consisting of phosphatidylcholine and lyso-phosphatidylcholine. The present method preferably comprises the administration of more than 0.05 g choline per day, preferably 0.1 to 2 g choline per day, more preferably 0.2 to 1 g choline per day, most preferably 0.2 to 0.5 g choline per day. The present composition or combination preferably comprises 0.05 to 2 g choline per 100 ml of the liquid product, preferably 0.2 to 1 g, more preferably up to 0.5 g choline per 100 ml. In one embodiment, the composition or combination preferably comprises 10-2000 mg choline per 100 kcal, more preferably at least 20, 30, 40, 50, 60, 70, 80, 90, 100 mg/100 kcal. The preferred upper limit for the above mentioned range is 2000, 1500, 1250, 1000 mg/100 kcal. The above numbers are based on choline, the amounts of choline equivalents or sources can be calculated taking the molar equivalent to choline into account, based on the molar mass of 104 g choline/mol.

### Phospholipids

Preferably, the method, composition and combination according to the present invention comprises phospholipids, preferably 0.1-50 weight % phospholipids based on total weight of lipids, more preferably 0.5-20 weight %, more preferably between 1 and 10% weight %, most preferably between 1 and 5 weight % based on total weight of lipids. The present method preferably comprises the administration of 50-1000 mg phospholipids. The total amount of lipids is preferably between 10 and 30 weight % on dry matter, and/or between 2 and 10 g lipid per 100 ml for a liquid product. The composition or combination preferably comprises between 0.01 and 1 gram lecithin per 100 ml, more preferably between 0.05 and 0.5 gram lecithin per 100 ml. A composition with these preferred amounts was found to be very effective. In one embodiment, the amount of phospholipids is between 0.01 and 0.5 g, more preferably between 0.05 and 0.25 g per 100 ml.

### B Vitamins

The method, combination and composition according to the present invention preferably comprise at least one B complex vitamin. The vitamin B is selected from the group of vitamin B1 (thiamine), vitamin B2 (riboflavin), vitamin B3 (niacin or niacinamide), vitamin B5 (panthotenic acid), vitamin B6 (pyridoxine, pyridoxal, or pyridoxamine, or pyridoxine hydrochloride), vitamin B7 (biotin), vitamin B9 (folic acid or folate), and vitamin B12 (various cobalamins). Functional equivalents are encompassed within these terms.

Preferably at least one, more preferably at least two B vitamins are selected from the group consisting of vitamin B6, vitamin B12 and vitamin B9, including their functional equivalents, preferably vitamins B6 and/or B12. Again, functional equivalents are encompassed within these terms.

The vitamin B is to be administered in an effective dose, which dose depends on the type of vitamin B used. As a rule of thumb, a suitable minimum or a maximum dose may be chosen based on known dietary recommendations, for instance as recommended by Institute of Medicine (IOM) of the U.S. National Academy of Sciences or by Scientific Committee on Food (a scientific committee of the EU), the information disclosed herein and optionally a limited amount of routine testing. A minimum dose may be based on the estimated average requirement (EAR), although a lower dose may already be effective. A maximum dose preferably does not exceed the tolerable upper intake levels (UL), as recommended by IOM.

If present or administered, the vitamin B6 is preferably present in an amount to provide a daily dosage in the range of 0.1 to 50 mg, in particular in the range of 0.5 to 10 mg, more in particular in the range of 0.5 to 5 mg. The present composition or combination preferably comprises 0.1 to 50 mg vitamin B6 per 100 ml (liquid) product, more preferably 0.5 to 10 mg vitamin B6 per 100 ml (liquid) product, more preferably 0.5 to 5 mg vitamin B6 per 100 ml (liquid) product.

If present or administered, the vitamin B12 is preferably present in an amount to provide a daily dosage in the range of 0.5 to 15 μg, in particular in the range of 1 to 10 μg, more in particular in the range of 1 to 5 μg. The present composition or combination preferably comprises 0.5-15 μg vitamin B12 per 100 ml (liquid) product, more preferably 1 to 10 μg vitamin B12 per 100 ml (liquid) product, more preferably 1 to 5 μg vitamin B12 per 100 ml (liquid) product. The term “vitamin B12” incorporates all cobalamin equivalents known in the art.

Throughout the application, the terms ‘folic acid’, ‘folate’ and ‘B9’ are used interchangeably. If present or administered, the vitamin B9 is preferably present in an amount to provide a daily dosage in the range of 0.05 to 1 mg, in particular in the range of 0.05 to 0.5 mg, preferably up to 0.4 mg, more preferably up to 0.3 mg, even more in particular in the range of 0.05 to 0.2 mg, preferably below 0.19 mg, below 0.18 mg, below 0.17 mg, below 0.16 mg, particularly 0.05-0.15 mg, most preferably up to 0.1 mg per day. The present composition or combination preferably comprises 0.05 to 1 mg folic acid per 100 g (liquid) product, more preferably 0.05 to 0.5 mg folic acid per 100 ml (liquid) product, preferably up to 0.4 mg, more preferably up to 0.3 mg, even more preferably 0.05 to 0.2 mg folic acid per 100 ml (liquid) product, preferably below 0.19 mg, below 0.18 mg, below 0.17 mg, below 0.16 mg, most preferably 0.05-0.15 mg, most preferably up to 0.1 mg folic acid per 100 ml product. Folates include folic acid, folinic acid, methylated, methenylated and formylated forms of folates, their salts or esters, as well as their derivatives with one or more glutamic acid, and all in either reduced or oxidized form.

If B vitamins are present, it is preferably vitamins B6 and/or B12.

### Vitamins C, E, Selenium

The method, combination and composition of the invention preferably involves at least one, preferably at least two, most preferably all of the antioxidants selected from the group consisting of selenium, vitamin C and vitamin E, and functional equivalents thereof. Selenium is the most preferred antioxidant.

Vitamin C, or a functional equivalent thereof, may be present or administered in an amount to provide a daily dosage in the range of 0.01 to 2 g, in particular in the range of 0.025 to 0.5 g, more in particular in the range of 0.04 to 0.15 g. In one embodiment, vitamin C, or a functional equivalent thereof, is given in an amount in the range of 0.025 to 2 g, in particular in the range of 0.04 to 0.5 g, more in particular in the range of 0.04 to 0.15 g per 100 ml of the (liquid) product.

Tocopherol and/or an equivalent thereof (i.e. a compound having vitamin E activity) may be present in an amount to provide a daily dosage in the range of 0.01 to 0.5 g, in particular in the range of 0.01 to 0.25 g, more in particular in the range of 0.02 to 0.1 g, particularly 0.025 to 0.05 g, to prevent oxidative damage resulting from dietary PUFA. In one embodiment, tocopherol and/or equivalent is present in an amount in the range of 0.01 to 0.5 g, in particular in the range of 0.01 to 0.25 g, more in particular in the range of 0.02 to 0.1 g, particularly 0.025 to 0.05 g per 100 ml of the product. The term “tocopherol and/or an equivalent thereof”, and ‘alpha-TE’, as used in this description, comprises tocopherols, tocotrienols, pharmaceutical and/or nutritional acceptable derivatives thereof and any combination thereof. The above numbers correspond to the amount of alpha-tocopherol, recognized in the art.

The present composition preferably contains selenium, because of its excellent antioxidant activity. The present method preferably provides the administration of a composition or a combination comprising between 0.01 and 5 mg selenium per 100 ml liquid product, preferably between 0.025 and 0.1 mg selenium per 100 ml liquid product. The amount of selenium administered per day is preferably more than 0.01 mg, more preferably 0.01 to 0.5 mg, most preferably at least 0.025 mg per day.

In one embodiment, the combination of the invention may comprise inositol and/or iodine, preferably at least inositol. The composition may comprise iron minerals.

In a preferred embodiment, the method, combination or composition of the invention preferably comprises (i) said ω-3 polyunsaturated fatty acid (LCPUFA) selected from the group consisting of docosahexaenoic acid (DHA), eicosapentaenoic acid (EPA) and docosapentaenoic acid (DPA); (ii) said one of more of uridine, cytidine and/or an equivalent thereof, including salts, phosphates, acyl derivatives and/or esters; and (iii) L-leucine, and further comprises at least one, more preferably at least 2, even more preferably at least 3, even more preferably at least 4 of vitamin D and/or vitamin K, including their functional equivalents; and at least one of vitamin C, vitamin E and selenium, including their equivalents preferably at least selenium. The method, combination or composition of the invention preferably comprises vitamin D and vitamin K, including equivalents thereof.

The composition of the invention is preferably a liquid nutrtional product comprising proteins, carbohydrates and lipids, preferably comprising 10-30 g, more preferably 15-25 g protein or protein equivalent per serving. The term ‘protein equivalent’ is an art-recognized term to express the amounts of the free amino acids as the amount of amino acids as if it was part of a protein, i.e. the weight value of amino acids is understood as the protein equivalent weight value, unless otherwise specified. The contribution of the amino acids to protein represents about 81% of the weight of the individual amino acids.

A serving is preferably 100-200 ml. Per serving, carbohydrates are preferably present in amounts of 10-25 g.

In a specific embodiment, the nutritional composition provides a complete nutrition, i.e. nutrition providing vitamins, minerals, and food energy in the form of carbohydrates, proteins, and fats in suitable amounts to provide a healthy nutritional intake. Advantageously the complete nutrition also contains dietary fibre and/or a probiotic. Suitable compositions for complete nutritions are known in the art, and the present nutritional composition can be based on Foods for Special Medical Purposes regulations (FSMP, EC Commission Directive 1999/21/EC, http://eur-lex.europa.eu/legal-content/EN/ALL?uri=CELEX:31999L0021), with the proviso that the nutritional composition (for use) according to the invention comprises at least the components as defined here above.

With the above restraints, the invention preferably pertains to a method, composition or combination as defined here above, comprising administering, per daily dosage or per 100 ml product, at least 3, 4, 5, 6, 7, 8, 9 or all of:
0.25 to 5 g, preferably 0.5 to 5 g, more preferably 1 to 2.5 g of DHA+EPA+DPA taken together;
0.05-5 g, preferably 0.1-2.5 g, more preferably 0.25-1 g of uridine;
0.1 to 2 g, preferably 0.2 to 1 g, more preferably 0.2 to 0.5 g choline;
0.05-0.6 g, preferably 0.05-0.6 g, more preferably 0.06-0.2 g phospholipids;
0.1 to 50 mg, preferably 0.5 to 10 mg, more preferably 0.5 to 5 mg of vitamin B6;
0.5 to 15 μg, preferably 1 to 10 82 g, more preferably 1 to 5 μg of vitamin B12;
0.05 to 0.5 mg, preferably 0.05 to 0.2 mg, preferably less than 0.19 mg, more preferably less than 0.18 mg, preferably less than 0.17 mg, more preferably less than 0.16 mg, more preferably 0.05 to 0.15 mg vitamin B9;
0.01 to 2 g, preferably 0.025 to 0.5 g, more preferably 0.04 to 0.15 g of Vitamin C;
0.01 to 0.5 g, preferably 0.01 to 0.25 g, more preferably 0.02 to 0.1 g of alpha-tocopherol; and
more than 0.01 mg, preferably 0.01 to 0.5 mg, more preferably at least 0.025 mg selenium.

The method, composition or combination may further comprise, per daily dosage or per 100 ml product:
1 to 100 μg, in particular in the range of 1 to 50 μg, preferably 2-25 μg, preferably at least 3 μg, more preferably at least 4 μg, even more preferably at least 5 μg, more in particular in the range of 5 to 25 μg, even more preferably at least 5-15 μg of vitamin D; and/or
1 to 100 μg, preferably 5 to 50 μg, preferably at least 7 μg, preferably at least 8 μg, more preferably at least 9 μg, even more preferably in the range of 10 to 50 μg, particularly at least 11, 12, 13, 14, 15 μg, preferably up to 40 μg, more preferably up to 30 μg of Vitamin K, preferably vitamin K1.

It is preferred that both vitamin D and vitamin K are present.

L-leucine is present in the aforementioned amounts, and preferably the sum of threonine, valine, histidine and methionine is at least 12 wt %, preferably at least 13 wt %, more preferably at least 14 wt %, preferably at least 15-60 wt %, more preferably 16-40 wt %, based on total proteinaceous matter.

More preferably, the composition comprises, per daily dose or preferably per 100 ml composition:
0.1-0.5 g, preferably 0.2-0.4 g EPA,
0.5-1.5 g, preferably 0.75-1 g DHA,
0.1-0.5 g, preferably 0.2-0.4 g choline,
0.05-0.5 g, preferably 0.06-0.2 g phospholipids,
0.25-0.8 g, preferably 0.4-0.7 g UMP (uridine monophosphate),
0.01-0.05 g, preferably 0.015-0.04 g vitamin E (alpha-TE),
0.04-0.1 g, preferably 0.04-0.09 g vitamin C,
0.035-0.08 mg, preferably 0.035-0.07 mg selenium,
1-5 preferably 2-4 μg vitamin B12,
0.5-3 mg, preferably 0.5-2 mg vitamin B6, and
0.05-0.2 mg, preferably less than 0.19 mg, more preferably less than 0.18 mg, preferably less than 0.17 mg, more preferably less than 0.16 mg, preferably 0.05-0.15 mg, more preferably 0.05-0.1 mg folic acid.

The composition may further comprise vitamin D and/or vitamin K in the aforementioned amounts. L-leucine is present in the aforementioned amounts and preferably the sum of threonine, valine, histidine and methionine is at least 12 wt %, preferably at least 13 wt %, more preferably at least 14 wt %, preferably at least 15-60 wt %, more preferably 16-40 wt %, based on total proteinaceous matter.

### EXAMPLES

### Example 1

### Exemplary Composition for use According to the Invention

| | invention formula |
|---|---|
| | (per serving or per day) |
| Energy (kcal) | 173.5 |
| Protein/Protein | 20 |
| Equivalent* (g) | |
| Carbohydrates (g) | 16.5 |
| Fat (g) | 3.05 |
| DHA (22:6n-3; mg) | 0.88 |
| Micronutrients* | |
| Calcium (mg) | 356 |
| Phosphorus (mg) | 276 |
| Vitamin E (mg α-TE) | 33.6 |
| Vitamin C (mg) | 44.4 |
| Vitamin D (μg) | 6 |
| Vitamin K (μg) | 20 |
| Folic acid (μg) | 160 |
| Niacin/Vitamin B3 (mg) | 7.1 |
| Vitamin B6 (mg) | 0.58 |
| Vitamin B12 (μg) | 1.8 |
| Choline (mg) | 153 |
| Nucleotides | |
| Uridine-5′- | 625 |
| monophosphate (mg) | |

*The term ‘protein equivalent’ is an art-recognized term to express the amounts of the free amino acids as the amount of amino acids as if it was part of a protein, i.e. the weight value of amino acids is understood as the protein equivalent weight value, unless otherwise specified. The contribution of the amino acids to protein represents about 81% of the weight of the individual amino acids.

### Experimental Evidence: Effect of the Combination According to the Invention on Equilibrioception in Heatlhy Mice

### Material and Methods

### Animals & Diets Used in the Experiment

SNC is the active supplement that was added to a basal animal chow. The supplement involved a combination of nutrients including uridine-5′-monophosphate, choline, and the omega-3 polyunsaturated fatty acids (LCPUFAs) docosahexaenoic acid (DHA) and eicosapentaenoic acid as it is represented in table 2 here below.

TABLE 2
SNC composition added to mice diet

| | Amount | |
|---|---|---|
| | per 100 gram | |
| component | of mice diet | |
| EPA | 200 | mg |
| DHA | 3000 | mg |
| Phospholipids | 755 | mg |
| Choline | 313 | mg |
| UMP | 1000 | mg |
| Vitamin E (alpha-TE) | 157 | mg |
| Vitamin C | 160 | mg |
| Selenium | 1.1 | μg |
| Vitamin B12 | 1.1 | μg |
| Vitamin B6 | 2.7 | mg |
| Folic acid | 700 | μg |

In this experiment, the BTBR Pahe^{cnu2} mouse model was used. Male and female wild-type (WT) littermates were obtained from our own breeding. Male and female mice were housed in separate rooms under the same 12:12 light/dark cycle, temperature and humidity conditions. 48 WT mice were subdivided into two experimental groups, receiving diets with and without SNC. The basal formula for all diets was AIN93G (Research Diet Services, Wijk bij Duurstede): (1) WT control; and (2) WT+SNC.

The levels of amino acids within the food are depicted in table 3. All diets met the minimal nutritional requirement for laboratory animals. Following weaning at P28, the animals were genotyped and allocated to the different groups. Starting at P31, the animals had ad libitum access to these diets and water for 12 weeks.

All animals were weighed daily between 16:00-18:00 in the first week of the experiment (PND 31-PND 38) and from then on weekly. Food intake was measured daily.

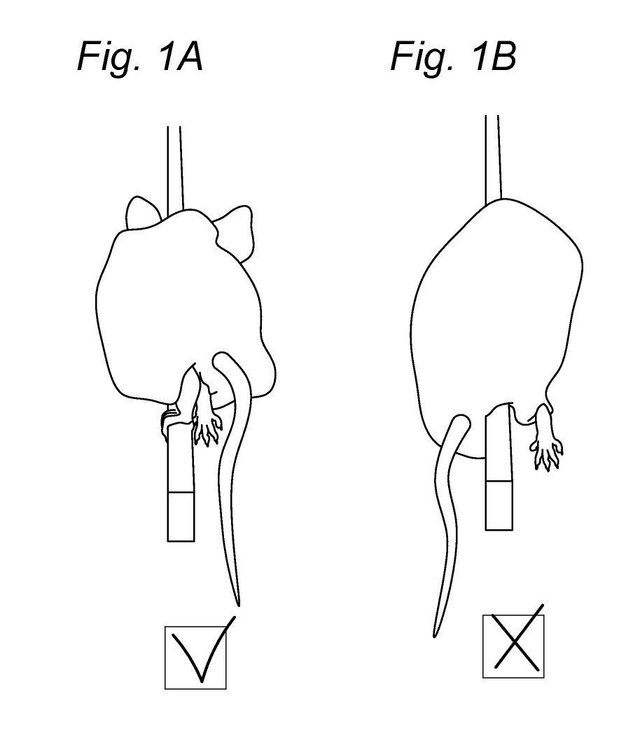

In week 16, all animals were subjected to the balance beam (experimental setup shown in FIG. 1A). This is a 100 cm long narrow beam that the mice cross to return to their home cage at the far end of the beam. The percentage of correct versus incorrect steps are calculated and shown in FIG. 1B. The percentage of correct steps on the balance beam was significantly higher in mice treated with the SNC-containing diet compared to control diet (p<0.05).

TABLE 3
Amino acid levels in the different nutritional
compositions of the treatment groups

| | g/100 g diet | g/100 g diet |
|---|---|---|
| | Control diet | SNC diet |
| Alanine | 0.33 | 0.33 |
| Arginine | 0.45 | 0.45 |
| Aspartic acid | 0.80 | 0.80 |
| Cystine | 0.24 | 0.24 |
| Glutamic acid | 2.55 | 2.55 |
| Glycine | 0.23 | 0.23 |
| Histidine | 0.33 | 0.33 |
| Isoleucine | 0.59 | 0.59 |
| Leucine | 1.09 | 1.09 |
| Lysine | 0.92 | 0.92 |
| Methionine | 0.33 | 0.33 |
| Phenylalanine | 0.62 | 0.62 |
| Proline | 1.43 | 1.43 |
| Serine | 0.67 | 0.67 |
| Threonine | 0.47 | 0.47 |
| Tryptophan | 0.16 | 0.16 |
| Tyrosine | 1.50 | 1.50 |
| Valine | 0.70 | 0.70 |

1.-18. (canceled)

19. A method for improving or promoting equilibrioception or coordination and balance in a healthy non-elderly human subject less than 50 years of age, comprising administering to the healthy subject a composition comprising:
(i) ω-3 polyunsaturated fatty acid (LCPUFA) selected from the group consisting of docosahexaenoic acid (DHA), eicosapentaenoic acid (EPA) and docosapentaenoic acid (DPA);
(ii) one of more of uridine, cytidine, deoxyuridine, uracil, citicoline and salts, phosphates, acyl derivatives and esters thereof;
(iii) proteinaceous matter comprising at least free leucine;
(iv) vitamins B6 and B12;
(v) choline, a choline salt and/or choline ester; and
(vi) an antioxidant selected from the group consisting of selenium, vitamin C and vitamin E.

20. The method according to claim 19, wherein the proteinaceous matter further comprises threonine, valine, histidine and methionine, wherein the sum of threonine, valine, histidine and methionine is at least 12 wt %, based on total proteinaceous matter.

21. The method according to claim 20, wherein the proteinaceous matter further comprises threonine, valine, histidine and methionine, which amino acids altogether are present in an amount between 15-60 wt %, based on total proteinaceous matter.

22. The method according to claim 19, wherein the total amount of L-leucine provided by the proteinaceous matter amounts to at least 7 wt %, based on total proteinaceous matter.

23. The method according to claim 22, wherein the total amount of L-leucine provided by the proteinaceous matter amounts to between 9-20 wt %, based on total proteinaceous matter.

24. The method according to claim 19, wherein the proteinaceous matter comprises less than 2 wt % phenylalanine, based on total proteinaceous matter.

25. The method according to claim 24, wherein the proteinaceous matter comprises less than 0.5 wt % based on total proteinaceous matter.

26. The method according to claim 19, wherein the proteinaceous matter comprises leucine, isoleucine and valine, which amino acids altogether are present in an amount of at least 15 wt %, based on total proteinaceous matter.

27. The method according to claim 19, wherein the proteinaceous matter comprises leucine, isoleucine and valine, which amino acids altogether are present in an amount between 15 and 35 wt %, based on total proteinaceous matter.

28. The method according to claim 19, wherein the composition further comprises vitamin D and/or vitamin K.

29. The method according to claim 19, wherein the composition comprises selenium, vitamin C and vitamin E.

30. A nutritional composition comprising:
(i) ω-3 polyunsaturated fatty acid (LCPUFA) selected from the group consisting of docosahexaenoic acid (DHA), eicosapentaenoic acid (EPA) and docosapentaenoic acid (DPA);
(ii) one of more of uridine, cytidine, deoxyuridine, uracil, citicoline and salts, phosphates, acyl derivatives and esters thereof;
(iii) proteinaceous matter comprising free leucine in a total amount of at least 7 wt %, based on total proteinaceous matter, and less than 2 wt % phenylalanine, based on total proteinaceous matter,
(iv) vitamins B6 and B12,
(v) choline, a choline salt and/or choline ester, and
(vi) antioxidants selected from the group consisting of selenium, vitamin C and vitamin E.

31. The composition according to claim 30, wherein the proteinaceous matter comprises free leucine in a total amount between 9-20 wt %, based on total proteinaceous matter.

32. The composition according to claim 30, further comprising threonine, valine, histidine and methionine, which amino acids altogether are present in an amount of at least 12 wt %, based on total proteinaceous matter.

33. The composition according to claim 32, further comprising threonine, valine, histidine and methionine, which amino acids altogether are present in an amount between 15-60 wt %, based on total proteinaceous matter.

34. The composition according to claim 30, comprising leucine, isoleucine and valine, which amino acids altogether are present in an amount of at least 15 wt %, based on total proteinaceous matter.

35. The composition according to claim 34, comprising leucine, isoleucine and valine, which amino acids altogether are present in an amount between 15 and 35 wt %, based on total proteinaceous matter.

36. The composition according to claim 30, further comprising vitamin D and/or vitamin K.
